(19) United States
(12) Patent Application Publication
(10) Pub. No.: US 2025/0252761 A1
(43) Pub. Date: Aug. 7, 2025

(54) CELL DETECTION DEVICE, CELL DIAGNOSIS SUPPORT DEVICE, CELL DETECTION METHOD, AND CELL DETECTION PROGRAM

(71) Applicants: KYOCERA COMMUNICATION SYSTEMS CO., LTD., Kyoto (JP); KYOTO PREFECTURAL PUBLIC UNIVERSITY CORPORATION, Kyoto (JP)

(72) Inventors: Kengo UENO, Kyoto (JP); Masaya NAKANISHI, Kyoto (JP); Kohei HENMI, Kyoto (JP); Masayuki HIGASHI, Kyoto (JP); Osamu UKIMURA, Kyoto (JP); Masatomo KANEKO, Kyoto (JP); Keisuke TSUJI, Kyoto (JP)

(21) Appl. No.: 18/853,302

(22) PCT Filed: Mar. 30, 2023
(86) PCT No.: PCT/JP2023/013072

§ 371 Date: Oct. 1, 2024

(30) Foreign Application Priority Data
Apr. 4, 2022 (JP) 2022-062650

(51) Int. Cl. G06V 20/69; G06T 7/00; G06V 10/25; G06V 10/56

(52) U.S. Cl. CPC G06V 20/698; G06T 7/0012; G06V 10/25; G06V 10/56; G06T 2207/30024

(57) ABSTRACT

A cell detection device detects a cell image in image data obtained by capturing an image of a slide sample prepared on the basis of liquid in which cells are dispersed. The cell detection device includes: an image data acquisition section that acquires a plurality of pieces of the image data that are different from each other in terms of resolution; a first detection section that detects a candidate region of the cell image in first image data; an identification section that identifies a region for the candidate region that is in second image data having higher resolution than the first image data; and a second detection section that detects the cell image from the region in the second image data. As a result, a cell contained in liquid excreted from a human body can be accurately and efficiently diagnosed.

### TECHNICAL FIELD

The present invention relates to a cell detection device, a cytology support device, a cell detection method, and a cell detection program.

### BACKGROUND ART

A technology for pathologically diagnose, with use of a microscopic image, a specimen that was acquired from a subject has been known (Patent Literature 1). Patent Literature 1 discloses achieving efficient and highly accurate pathological determination by: classifying, into each group by data pattern, the microscopic image of sample tissue; and making pathological determination within the group. Patent Literature 2 discloses a dual detection method in which detection is performed using fluorescence imaging and staining of a cancer cell in body cavity fluid or urine.

### CITATION LIST

### Patent Literature

### [Patent Literature 1]

Japanese Patent Application Publication Tokukai No. 2010-203949

### [Patent Literature 2]

WO 2016/047676 A1

### SUMMARY OF INVENTION

### Technical Problem

Pathological diagnosis using a cell in liquid, such as urine, which is excreted from a human body, is a relatively less invasive examination and places less stress on a subject, and is thus preferable. However, urine contains, in addition to the cell subjected to pathological diagnosis, a cell that does not contribute to the diagnosis and a non-cellular component. Therefore, mechanical diagnosis on a cell that does not contribute to the diagnosis and that is present in a slide sample prepared on the basis of liquid excreted from a human body may result in decrease in accuracy of the diagnosis.

The technique disclosed in Patent Literature 1 is for performing pathological diagnosis on sample tissue, but is neither for use in cytology nor for diagnosis using, as a specimen, liquid excreted from a human body. On this account, the technique disclosed in Patent Literature 1 cannot accurately diagnose a slide sample that is prepared for cytology from liquid excreted from a human body. On the other hand, a technique disclosed in Patent Literature 2 is therefore complicated since this technique requires each of fluorescence imaging treatment and staining treatment on a cell in the body cavity fluid or urine.

In order to solve the above problems, the present invention has been attained. An object of an aspect of the present invention is to provide a technology for supporting accurate and efficient diagnosis on a cell that is contained in liquid excreted from a human body.

### Solution to Problem

In order to solve the above problems, a cell detection device in accordance with an aspect of the present invention is a cell detection device for detecting a cell image in image data obtained by capturing an image of a slide sample prepared on the basis of liquid in which cells are dispersed, the cell detection device including: an acquisition section that acquires a plurality of pieces of the image data that are different from each other in terms of resolution; a first detection section that detects a first cell region which indicates a candidate region of the cell image in first image data; an identification section that identifies a second cell region that is in second image data having higher resolution than the first image data and that corresponds to the first cell region; and a second detection section that detects the cell image from the second cell region in the second image data.

A cytology support device in accordance with an aspect of the present invention includes an evaluation section that evaluates the cell image detected by using the cell detection device in accordance with an aspect of the present invention.

In order to solve the above problems, a cell detection method in accordance with an aspect of the present invention is a cell detection method for detecting a cell image in image data obtained by capturing an image of a slide sample prepared on the basis of liquid in which cells are dispersed, the cell detection method including the steps of: acquiring a plurality of pieces of the image data that are different from each other in terms of resolution; detecting a first cell region which indicates a candidate region of the cell image in first image data; identifying a second cell region that is in second image data having higher resolution than the first image data and that corresponds to the first cell region; and detecting the cell image from the second cell region in the second image data.

A cell detection device in accordance with each aspect of the present invention can be realized by a computer. In this case, the present invention encompasses, in its technical scope, (i) a control program of the cell detection device for causing the computer to realize the cell detection device by causing the computer to operate as each section (software element) included in the cell detection device and (ii) a computer-readable storage medium in which the control program of the cell detection device is stored.

### Advantageous Effects of Invention

An aspect of the present invention can provide a technology for supporting accurate and efficient diagnosis on a cell that is contained in liquid excreted from a human body.

### BRIEF DESCRIPTION OF DRAWINGS

FIG. 1 is a block diagram illustrating a main configuration of a cytology support system that includes a cell detection device and a cytology support device in accordance with an aspect of the present invention.

FIG. 2 is a diagram schematically illustrating a cell detection process carried out by the cell detection device in accordance with an aspect of the present invention.

FIG. 3 is a flowchart showing a flow of the cell detection process in the cell detection device in accordance with an aspect of the present invention.

FIG. 4 is a flowchart showing a flow of a cell evaluation process in the cytology support device in accordance with an aspect of the present invention.

### DESCRIPTION OF EMBODIMENTS

### [Cytology Support System]

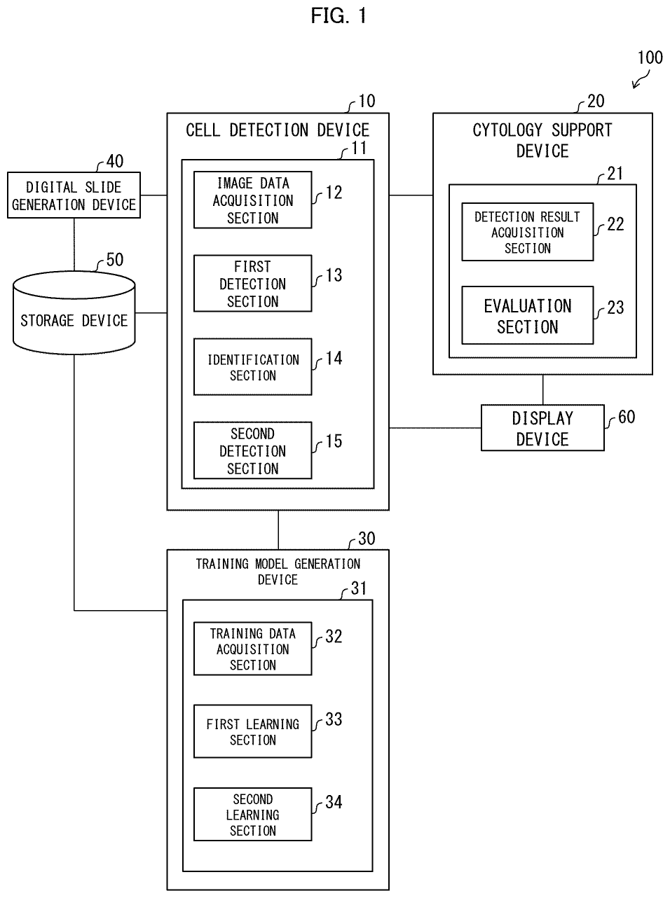

The following description will discuss a cytology support system in accordance with an aspect of the present invention, with reference to FIG. 1. FIG. 1 is a block diagram illustrating a main configuration of a cytology support system 100 that includes a cell detection device 10 and a cytology support device 20 in accordance with an aspect of the present invention.

The cytology support system 100 supports evaluation of a cell that is included in a specimen acquired from a subject. The cytology support system 100 thus supports cytology as to whether or not a subject is affected with a specific disease. A specimen acquired from a subject who is affected with a disease such as cancer may contain an abnormal cell, like a cancer cell, that have different appearance from a normal cell. In an example, the cytology support system 100 supports cytology by supporting evaluation as to whether or not such a specimen contains an abnormal cell.

In the cytology support system 100, the specimen subjected to cytology is liquid acquired from a subject. The specimen is preferably acquired by a method that is less invasive to a human body, and is preferably liquid, like urine, that is excreted from a human body. Further, the specimen may be a body cavity fluid like pleural effusion or may be a storage solution in which cells acquired from a human body is stored, such as a specimen to be used in liquid-based cytology of cervical cancer. In the cytology support system 100, a type of cells subjected to cytology is not particularly limited, but is preferably cells that are separated into each individual cell in the specimen. In a case where the cells in the specimen are in a cell mass state, a cell separation process may be carried out on the cells.

In the specimen, cells that are subjected to evaluation for cytology (cells that contribute to cytology) are dispersed. Further, in the specimen, cells that are not subjected to evaluation for cytology (cells that do not contribute to cytology) and non-cellular components are also dispersed. An image that is obtained by capturing an image of the specimen include (a) a cell image of a cell that contributes to cytology and (b) an image of a cell that does not contribute to cytology and an image of a non-cellular components. The cytology support system 100 accurately and efficiently distinguishes between (a) the cell image of the cell that contributes to cytology and (b) the image of the cell that does not contribute to cytology and the image of the non-cellular component. In this way, the cytology support system 100 accurately and efficiently detects the cell image that contributes to cytology, and this supports cytology using the cell image.

The cytology support system 100 includes the cell detection device 10 and the cytology support device 20. The cytology support system 100 further includes a training model generation device 30, a digital slide generation device 40, a storage device 50, and a display device 60. The cytology support system may be a device which integrally includes these devices, or may include these devices as separate devices.

The digital slide generation device 40 generates a digital slide of a slide sample prepared on the basis of the liquid in which cells are dispersed. The digital slide generation device 40 can be a well-known device that generates a digital slide by a well-known method. The digital slide generation device 40 generates a digital slide with use of a slide sample prepared on the basis of a specimen. For example, the digital slide is an image file in Pyramid Tiled Tiff format which includes a plurality of pieces of data having different magnification ratios such as 5 times, 10 times, 20 times, and 40 times in series. For example, a piece of image data having a magnification ratio of 5 times is a rough image having low resolution, and a piece of data having a magnification ratio of 40 times is a sharp image having high resolution.

The slide sample for generating the digital slide is prepared on the basis of a specimen that is liquid in which cells are dispersed. The slide sample can be generated, for example, by applying, to a glass slide, the liquid in which the cells are dispersed. The specimen of the slide sample is stained by appropriate staining in accordance with a type of a cell, a type of disease to be diagnosed, and/or the like.

The digital slide generated by the digital slide generation device 40 is sent to the cell detection device 10 or the storage device 50, and can be used for supporting cytology in the cytology support system 100. Note that the cytology support system 100 may acquire a digital slide that was generated by another device, and use the digital slide for supporting cytology.

The storage device 50 stores a program and data that are to be used in the cytology support system 100. The storage device 50 stores, for example, the digital slide which was generated in the digital slide generation device 40. Further, the storage device 50 may store a digital slide which was generated in another device. Furthermore, the storage device 50 stores, for example, training data to be used in generation of a training model in the training model generation device 30 and a training model generated in the training model generation device 30. In addition, the storage device 50 stores, for example, a training model, input information and output information for use in detection of a cell in the cell detection device 10. The storage device 50 may have, on cloud or in a server, a database in which various data are stored.

The display device 60 displays a detection result of the cell detection device 10 or an evaluation result of the cytology support device 20. Further, the display device 60 may output a diagnosis result based on the evaluation result of the cell which was obtained by the cytology support device 20. Here, the evaluation result of the cell is intended to refer to, for example, whether the cell is evaluated to be positive or negative with reference to a predetermined reference. Furthermore, the diagnosis result based on the evaluation result of the cell is intended to refer to a diagnosis that the cell is affected with a specific disease in a case where the cell is evaluated to be positive or refer to a diagnosis that the cell is not affected with a specific disease in a case where the cell is evaluated to be negative.

The display device 60 may be, for example, a PC display, or a display of a mobile device such as a smartphone. Further, the cytology support system 100 may include a printing device that prints the content displayed on the display device 60.

### (Cell Detection Device 10)

The cell detection device 10 is a device for detecting a cell image in image data that was obtained by capturing an image of a slide sample prepared on the basis of liquid in which cells are dispersed. The cell detection device 10 detects, with use of digital slide data obtained from the slide sample, the cell image of the cell that contributes to cytology in a specimen that is the liquid in which the cells are dispersed. The cell detection device 10 accurately and efficiently detects the cell image of the cell that contributes to cytology, by distinguishing between (a) the cell that contributes to cytology in the spacemen and (b) the cell that does not contribute to cytology and the non-cellular component in the specimen.

The cell detection device 10 includes a control section 11. The control section 11 is to perform overall control of each section of the cell detection device 10, and is realized by, for example, a processor and a memory. In this example, the processor accesses a storage (not illustrated) and load, to the memory, a program (not illustrated) stored in the storage, and executes a series of commands included in the program. As a result, each section of the control section 11 is configured. As such sections, the control section 11 includes an image data acquisition section (acquisition section) 12, a first detection section 13, an identification section 14, and a second detection section 15.

The image data acquisition section 12 acquires a plurality of pieces of image data which are different from each other in terms of resolution and which are obtained by capturing an image of a slide sample prepared on the basis of liquid in which cells are dispersed. The image data acquired by the image data acquisition section 12 is image data that is included in digital slide data constituted by the plurality of pieces of the image data which are different from each other in terms of resolution and which are obtained by capturing the image of the slide sample. The image data acquisition section 12 acquires first image data that is low-resolution image data having low resolution and second image data that is high-resolution image data having high resolution. In other words, the second image data has higher resolution than the first image data. For example, the first image data has a resolution of not less than 10000×5000 (length by width) pixels and not more than 50000×25000 (length by width) pixels; and the second image data has a resolution of not less than 50000×25000 (length by width) pixels and not more than 250000×125000 pixels. The image data acquisition section 12 outputs, among the image data having acquired, the first image data to the first detection section 13 and the second image data to the identification section 14.

The first detection section 13 detects a first cell region which indicates a candidate region of the cell image in the first image data. The first detection section 13 detects, in the first image data having low resolution, the first cell region which is a candidate region of a cell image of interest. The first detection section 13 performs primary filtering for detecting the cell image. The first detection section 13 outputs, to the identification section 14, information that indicates the first cell region detected.

The first detection section 13 detects the first cell region according to a result of comparing, with a predetermined reference, size and/or color thickness of the candidate region in the first image data. The first detection section 13 detects the first cell region by using, as a condition of the primary filtering, the size of the cell image and/or the color thickness of the cell image. This allows the first detection section 13 to reduce noise such as the cell that does not contribute to cytology and the non-cellular component in the image data.

The first detection section 13 may: input the first image data as input information to a first image analysis algorithm configured to (i) receive, as the input information, the image data obtained by capturing the image of the slide sample prepared on the basis of the liquid in which the cells are dispersed, and (ii) output an inference result regarding the candidate region of the cell image in the image data; and detects, as the first cell region, an outputted inference result of the candidate region of the cell image.

The first image analysis algorithm is designed to detect the first cell region in the first image data. Examples of the first image analysis algorithm include a machine learning model, a trained model in which a hyperparameter(s) is/are set, and a rule-based model in which a feature(s) and a parameter(s) are set. Details of the first image analysis algorithm will be described later.

The identification section 14 identifies a second cell region that is in the second image data having higher resolution than the first image data and that corresponds to the first cell region. The identification section 14 can identify the second cell region by converting coordinates of the first cell region in the first image data to coordinates of corresponding region in the second image data. Such coordinate conversion that is carried out by the identification section 14 can be carried out by a well-known conversion method such as linear conversion. The identification section 14 outputs, to the second detection section 15, information that indicates the second cell region identified.

The second detection section 15 detects a cell image from the second cell region in the second image data. The second detection section 15 detects a cell image of interest from the second cell region which is the candidate region that has been narrowed down by performing the primary filtering with use of the first image data having low resolution. The second detection section 15 performs secondary filtering for detecting the cell image. The second detection section 15 outputs detected cell image data, to the cytology support device 20, the storage device 50, or the display device 60.

The second detection section 15 detects the cell image according to a result of comparing, with a predetermined reference, at least one selected from the group consisting of the presence or absence of a cell nucleus in the second cell region, size of the cell nucleus, a degree of defocus, and a degree of overlap of cell regions. The second detection section 15 detects the cell image by using, as a secondary filtering condition, at least one selected from the group consisting of the presence or absence of the cell nucleus, the size of the cell nucleus, the degree of defocus, and the degree of overlap of cell regions. This allows the second detection section 15 to exclude not only noise such as a cell that does not contribute to cytology and a non-cellular component in the image data but also an image that is not suitable for evaluation because of being out of focus.

The second detection section 15 may: input, as input information, the information that indicates the second cell region to a second image analysis algorithm configured to (i) receive, as the input information, information that indicates the candidate region of the cell image, and (ii) output an inference result regarding the cell image in the candidate region; and detects, as the cell image, an outputted inference result of the cell image.

The second image analysis algorithm is designed to detect the cell image in the second cell region. Examples of the second image analysis algorithm include a machine learning model, a trained model in which a hyperparameter(s) is/are set, and a rule-based model in which a feature(s) and a parameter(s) are set. Details of the second image analysis algorithm will be described later.

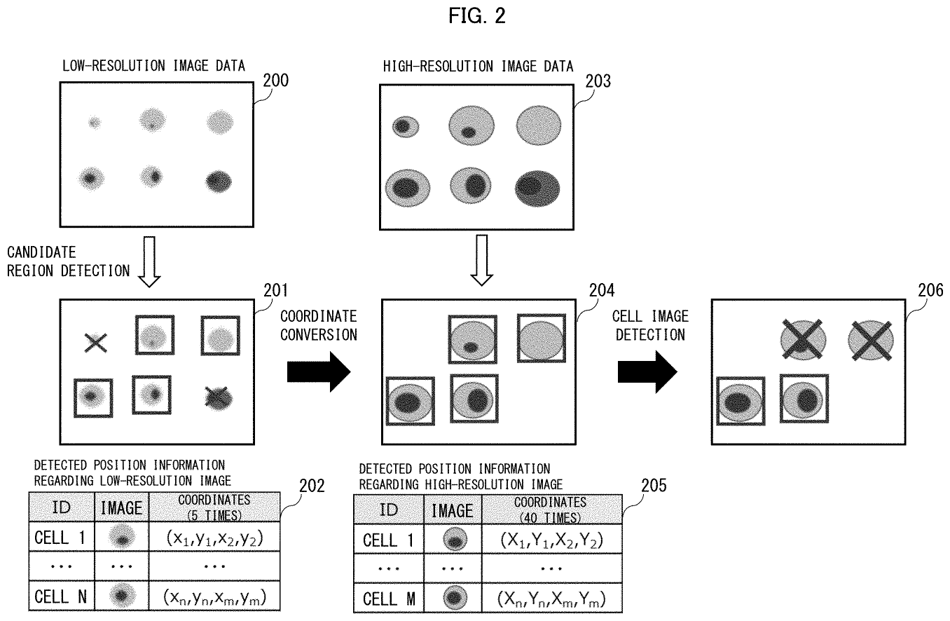

The following description will discuss an overview of a cell detection process using the cell detection device 10 as described above, with reference to FIG. 2. FIG. 2 is a diagram schematically illustrating a cell detection process carried out by the cell detection device 10 in accordance with an aspect of the present invention.

As illustrated in FIG. 2, in the cell detection device 10, the image data acquisition section 12 acquires a low-resolution image 200 and a high-resolution image 203. The first detection section 13 detects, in the low-resolution image 200, the first cell region which is a candidate region of the cell image. The first detection section 13 may put a box around the first cell region detected, as illustrated in a low-resolution image 201.

The identification section 14 acquires coordinate data 202 of each region, as position information of a region which is enclosed by the box and which is illustrated in the low-resolution image 201. Then, the identification section 14 converts the coordinate data 202 to coordinate data in the high-resolution image 203. The identification section 14 may put, on the basis of converted coordinate data 205, a box around the second cell region corresponding to the first cell region, as illustrated in a high-resolution image 204. The second detection section 15 detects the cell image from the second cell region illustrated in the high-resolution image 204, and distinguishes between the cell image and the other images as illustrated in a high-resolution image 206.

In this way, in the cell detection device 10, in addition to reducing noise by first performing the primary filtering with use of a low-resolution image, a cell image is detected by performing the secondary filtering with use of a high-resolution image. Therefore, it is possible to detect the cell image in a shorter period of time than detecting the cell image with use of digital slide data having a very large data size, and further, use of an analysis device having a large processing capacity is not necessary. In addition, since noise is removed by performing filtering two times, it is possible to accurately detect the cell image. In other words, with the cell detection device 10, it is possible to carry out accurate and efficient detection of the cell image.

In cytology, a cell subjected to diagnosis is detected from a specimen, and for example, with regard to a cell image, whether the cell is positive or negative is determined with respect to a predetermined reference. However, it is a large burden to an operator to detect a target cell from a specimen in which various cells and non-cellular components are present in a mixed manner. Further, in cytology, variation may occur depending on a level of skill of a pathologist or a cytotechnologist or test equipment. This may affect accuracy of the diagnosis. According to the cell detection device 10, a cell is detected by an image analysis algorithm or the like. Therefore, variation caused by an operator or an operation environment is unlikely to occur.

Further, in cytology, since a digital slide generated from a slide sample has a very large data size, an analysis device having a large processing capacity is necessary. In addition, use of a high-resolution image is essential for accurate cytology. However, processing a high-resolution image takes both time and labor. In the cell detection device 10, after noise is reduced with use of a low-resolution image, a candidate of a cell image is detected. Thereafter, a cell is detected with use of a high-resolution image. This makes it possible to accurately and efficiently detect the cell.

### (Cytology Support Device 20)

The cytology support device 20 is a device that supports cytology using a cell image(s). The cytology support device 20 performs cytology with use of a cell image which is a detection result of the cell detection device 10. In the cell image detected by the cell detection device 10, (a) a cell that contributes to cytology and (b) a cell that does not contribute to cytology and a non-cellular component are accurately and efficiently distinguished from each other. Accordingly, less noise exists. Therefore, the cytology support device 20 can support cytology by accurate and efficient evaluation of a cell with use of a cell image with less noise.

The cytology support device 20 includes a control section 21. The control section 21 is to perform overall control of each section of the cytology support device 20, and is realized by, for example, a processor and a memory. In this example, the processor accesses a storage (not illustrated) and load, to the memory, a program (not illustrated) stored in the storage, and executes a series of commands included in the program. As a result, each section of the control section 21 is configured. As such sections, the control section 21 includes a detection result acquisition section 22, and an evaluation section 23.

The detection result acquisition section 22 acquires, from the cell detection device 10, a cell image which is a detection result. The detection result acquisition section 22 may acquire a cell image which has been detected in advance and stored in the storage device 50. The detection result acquisition section 22 outputs, to the evaluation section 23, the cell image which has been thus acquired.

The evaluation section 23 evaluates the cell image. In an example, the evaluation section 23 determines, with regard to the cell image, whether a cell is positive or negative with reference to a predetermined reference. This allows the evaluation section 23 to perform cytology to determine that the cell is affected with a specific disease, in a case where the cell is positive, or determine that the cell is not affected with a specific disease, in a case where the cell is negative. Further, the evaluation section 23 may evaluate the cell image as to whether the cell is malignant or benign, or may evaluate a degree of progression (stage) of canceration of the cell.

The evaluation section 23 may: input the cell image as input information to a third image analysis algorithm configured to (i) receive, as the input information, information that indicates the cell image, and (ii) output an inference result of evaluation of the cell; and uses, as an evaluation of the cell image, an outputted inference result of the evaluation of the cell.

The third image analysis algorithm is designed to evaluate the cell image. Examples of the third image analysis algorithm include a machine learning model, a trained model in which a hyperparameter(s) is/are set, and a rule-based model in which a feature(s) and a parameter(s) is set. Details of the third image analysis algorithm will be described later.

The cytology support device 20 can support cytology by accurate and efficient evaluation of a cell, since the cytology support device 20 uses a cell image with less noise that has been detected by the cell detection device 10.

### (Training Model Generation Device 30)

The training model generation device 30 generates the first image analysis algorithm and the second image analysis algorithm which are used in the cell detection device 10, and generates the third image analysis algorithm which is used in the cytology support device 20.

The training model generation device 30 includes a control section 31. The control section 31 is to perform overall control of each section of the training model generation device 30, and is realized by, for example, a processor and a memory. In this example, the processor accesses a storage (not illustrated) and load, to the memory, a program (not illustrated) stored in the storage, and executes a series of commands included in the program. As a result, each section of the control section 31 is configured. As such sections, the control section 31 includes a training data acquisition section 32, a first learning section 33, a second learning section 34, and a third learning section 35.

The training data acquisition section 32 acquires training data for generating an image analysis algorithm. The training data acquisition section 32 reads the training data stored in the storage device 50, and outputs the training data to the first learning section 33, the second learning section 34, or the third learning section 35.

The training data for generating the first image analysis algorithm can be data in which (a) image data obtained by capturing an image of a slide sample prepared on the basis of liquid in which cells are dispersed and (b) information indicating a first cell region which is a candidate region of a cell image in the cell image data are associated with each other. The image data can be first image data having low resolution. Further, the training data for generating the first image analysis algorithm can be data that indicates determination criteria that are for detecting the first cell region and that have been set in advance. Such determination criteria can include features and parameters which are set for detecting the first cell region.

The training data for generating the second image analysis algorithm can be data in which (a) information indicating the candidate region of the cell image and (b) the cell image in the candidate region are associated with each other. The information indicating the candidate region can be information that indicates a second cell region corresponding to the candidate region in second image data having high resolution. Further, the training data for generating the second image analysis algorithm can be data that indicates determination criteria that are for detecting the cell image and that have been set in advance. Such determination criteria can include features and parameters which are set for detecting the cell image

The training data for generating the third image analysis algorithm can be data in which (a) cell image data and (b) an evaluation of a cell are associated with each other. The cell image data can be cell image data that was detected by the cell detection device 10. Further, the training data for generating the third image analysis algorithm can be data that indicates determination criteria that are for detecting the cell image and that have been set in advance. Such determination criteria can include features and parameters which are set for evaluating the cell image.

The first learning section 33 generates the first image analysis algorithm. Examples of the first image analysis algorithm include a machine learning model and a rule-based model. In a case where the first image analysis algorithm is a machine learning model, the first learning section 33 performs machine learning using the training data for generating the first image analysis algorithm, and generates the machine learning model as the first image analysis algorithm. The machine learning model may be subjected to hyperparameter tuning in accordance with inference accuracy. In a case where the first image analysis algorithm is a rule-based model, the first learning section 33 generates a rule-based model in which the determination criteria for detecting the first cell region are set. These training models can be each a first cell region detection model for detecting the first cell region in the first image data. The first learning section 33 stores, in the storage device 50, the first cell region detection model generated. The first learning section 33 may outputs, to the cell detection device 10, the first cell region detection model generated.

The second learning section 34 generates the second image analysis algorithm. Examples of the second image analysis algorithm include a machine learning model and a rule-based model. In a case where the second image analysis algorithm is a machine learning model, the second learning section 34 performs machine learning using the training data for generating the second image analysis algorithm, and generates the machine learning model as the second image analysis algorithm. The machine learning model may be subjected to hyperparameter tuning in accordance with inference accuracy. In a case where the second image analysis algorithm is a rule-based model, the second learning section 34 generates a rule-based model in which the determination criteria that are for detecting the cell image and that have been set in advance are set. These training models can be each a cell image detection model for detecting a cell image in the candidate region of the cell image. The second learning section 34 stores, in the storage device 50, the cell image detection model generated. The second learning section 34 stores, in the cell detection device 10, the cell image detection model generated.

The third learning section 35 generates the third image analysis algorithm. Examples of the third image analysis algorithm include a machine learning model and a rule-based model. In a case where the third image analysis algorithm is a machine learning model, the third learning section 35 performs machine learning using the training data for generating the third image analysis algorithm, and generates the machine learning model as the third image analysis algorithm. The machine learning model may be subjected to hyperparameter tuning in accordance with inference accuracy. In a case where the third image analysis algorithm is a rule-based model, the third learning section 35 generates a rule-based model in which the determination criteria that are for evaluating the cell image and that have been set in advance are set. These training models can be each a cell evaluation model for evaluating a cell in a cell image. The third learning section 35 stores, in the storage device 50, the cell evaluation model generated. The third learning section 35 outputs, to the cytology support device 20, the cell evaluation model generated.

In a case where the machine learning model is to be generated, the first learning section 33, the second learning section 34, and the third learning section 35 employ a known machine learning method such as a neural network, a decision tree, a random forest, a support vector machine, or the like.

### (Cell Detection Process)

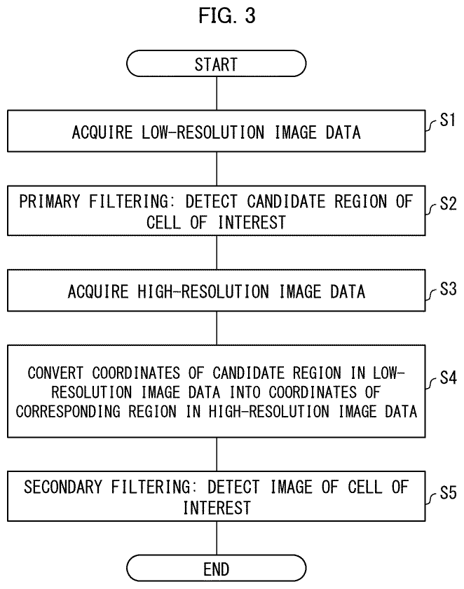

The following description will discuss a flow of a cell detection process (cell detection method) using the cell detection device 10, with reference to FIG. 3. FIG. 3 is a flowchart showing the flow of the cell detection process in the cell detection device 10 in accordance with an aspect of the present invention.

As shown in FIG. 3, first, the image data acquisition section 12 acquires low-resolution image data that has lower resolution among a plurality of pieces of image data which are different from each other in terms of resolution (step S1, acquisition step). Then, the first detection section 13 detects, in the low-resolution image, a candidate region (first cell region) of a cell image that contributes to cytology in the low-resolution image data, and performs primary filtering (step S2, detection step).

Next, the image data acquisition section 12 acquires high-resolution image data that has higher resolution (step S3). Then, the identification section 14 converts coordinates of the candidate region in the low-resolution image data to coordinates of a corresponding region in the high-resolution image data, and identifies, in the high-resolution image data, a second cell region that corresponds to the first cell region (step S4, identification step).

The second detection section 15 detects, from the second cell region in the high-resolution image data, the cell image of a cell that contributes to cytology (step S5, detection step), and outputs, to the cytology support device 20, data of the cell image as a detection result and ends the cell detection process.

### (Cell Evaluation Process)

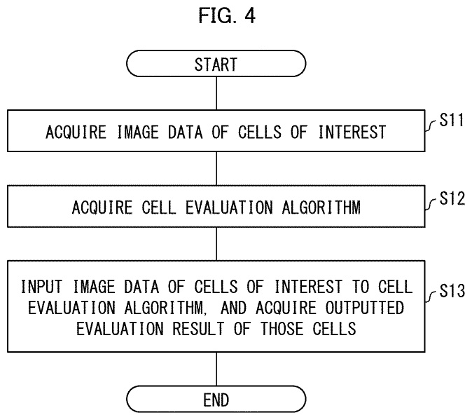

The following description will discuss a flow of a cell evaluation process using the cytology support device 20, with reference to FIG. 4. FIG. 4 is a flowchart showing the flow of the cell evaluation process in the cytology support device 20 in accordance with an aspect of the present invention.

As illustrated in FIG. 4, first, the detection result acquisition section 22 acquires cell image data of a cell that was detected by the cell detection device 10 and that contribute to cytology (step S11). Next, the evaluation section 23 acquires a cell evaluation algorithm which has been stored in the storage device 50 (step S12). Then, the evaluation section 23 inputs the cell image data to the cell evaluation algorithm, and acquires an outputted result of evaluation of the cell (step S13). Then, the evaluation section 23 outputs the result of evaluation to the display device 60, and ends the process.

### [Software Implementation Example]

A cell detection program for causing a computer to function as the cell detection device 10 and a cytology support program for causing a computer to function as the cytology support device 20 are included in the scope of the present invention.

Functions of the cell detection device 10 and the cytology support device 20 can be realized by a program that is for causing a computer to function as each of these devices and that causes the computer to function as each control block (in particular, each section included in the control section 11 and the control section 21) of these devices.

In this case, each of the above devices includes, as hardware for executing the above program, a computer having at least one control device (e.g., a processor) and at least one storage device (e.g., memory). Each function described in the above embodiments is realized by executing the program with use of the control device and the storage device.

The program can be stored in one or a plurality of non-transitory computer-readable storage media. Such a storage medium may be or may not be provided in the above-described device. In the latter case, the program can be made available to the device via any transmission medium (that is wired or wireless).

Further, one or all of the above-described functions of respective control blocks can be realized by a logic circuit. For example, an integrated circuit in which a logic circuit that functions as each of the control blocks is formed is also included in the scope of the present invention. Further, the function of each of the control blocks can be realized by a quantum computer.

Further, each of the processes which are described in the foregoing embodiments may be executed by artificial intelligence (AI). In this case, the AI may be operated by the above control device or alternatively, by another device (for example, an edge computer or the cloud server).

The present invention is not limited to the embodiments, but can be altered by a skilled person in the art within the scope of the claims. The present invention also encompasses, in its technical scope, any embodiment derived by combining technical means disclosed in differing embodiments.

### REFERENCE SIGNS LIST

10 cell detection device
12 image data acquisition section (acquisition section)
13 first detection section
14 identification section
15 second detection section
20 cytology support device
23 evaluation section

1. A cell detection device for detecting a cell image in image data obtained by capturing an image of a slide sample prepared on the basis of liquid in which cells are dispersed, the cell detection device comprising:
an acquisition section that acquires a plurality of pieces of the image data that are different from each other in terms of resolution;
a first detection section that detects a first cell region which indicates a candidate region of the cell image in first image data;
an identification section that identifies a second cell region that is in second image data having higher resolution than the first image data and that corresponds to the first cell region; and
a second detection section that detects the cell image from the second cell region in the second image data.

2. The cell detection device according to claim 1, wherein the first detection section detects the first cell region according to a result of comparing, with a predetermined reference, at least one selected from the group consisting of size and color thickness of the candidate region in the first image data.

3. The cell detection device according to claim 1, wherein the second detection section detects the cell image according to a result of comparing, with a predetermined reference, at least one selected from the group consisting of presence or absence of a cell nucleus in the second cell region, size of the cell nucleus, a degree of defocus, and a degree of overlap of cell regions.

4. The cell detection device according to claim 1, wherein the first detection section: inputs the first image data as input information to a first image analysis algorithm configured to (i) receive, as the input information, the image data obtained by capturing the image of the slide sample prepared on the basis of the liquid in which the cells are dispersed, and (ii) output an inference result regarding the candidate region of the cell image in the image data; and detects, as the first cell region, an outputted inference result of the candidate region of the cell image.

5. The cell detection device according to claim 1, wherein the second detection section: inputs, as input information, information that indicates the second cell region to a second image analysis algorithm configured to (i) receive, as the input information, information that indicates the candidate region of the cell image, and (ii) output an inference result regarding the cell image in the candidate region; and detects, as the cell image, an outputted inference result of the cell image.

6. A cytology support device comprising an evaluation section that evaluates the cell image detected by using the cell detection device recited in claim 1.

7. The cytology support device according to claim 6, wherein the evaluation section: inputs the cell image as input information to a third image analysis algorithm configured to (i) receive, as the input information, information that indicates the cell image, and (ii) output an inference result of evaluation of the cell; and uses, as an evaluation of the cell image, an outputted inference result of the evaluation of the cell.

8. A cell detection method for detecting a cell image in image data obtained by capturing an image of a slide sample prepared on the basis of liquid in which cells are dispersed, the cell detection method comprising the steps of:
acquiring a plurality of pieces of the image data that are different from each other in terms of resolution;
detecting a first cell region which indicates a candidate region of the cell image in first image data;
identifying a second cell region that is in second image data having higher resolution than the first image data and that corresponds to the first cell region; and
detecting the cell image from the second cell region in the second image data.

9. A non-transitory storage medium in which a cell detection program for causing a computer to function as the cell detection device recited in claim 1.
